(19) United States
(12) Patent Application Publication
(10) Pub. No.: US 2020/0072711 A1
(43) Pub. Date: Mar. 5, 2020

(54) FLUORESCENT ENDOPLASMIC RETICULUM TRACKERS FOR LIVE CELL IMAGING OF PATHOGENIC YEAST

(71) Applicant: Ramot at Tel Aviv University Ltd., Tel Aviv (IL)

(72) Inventors: Micha Fridman, Tel Aviv (IL); Raphael I. Benhamou, Tel Aviv (IL); Ido M. Herzog, Tel Aviv (IL); Qais Z. Jaber, Tel Aviv (IL)

(21) Appl. No.: 16/554,360

(22) Filed: Aug. 28, 2019

Related U.S. Application Data
(60) Provisional application No. 62/723,707, filed on Aug. 28, 2018.

(51) Int. Cl. G01N 1/30; G01N 33/569; G01N 33/58; C09K 11/06; C09B 62/343

(52) U.S. Cl. CPC G01N 1/30; G01N 33/56961; G01N 33/582; G01N 2021/6439; C09B 62/3435; G01N 2001/302; C09K 11/06

(57) ABSTRACT

Particular 1,2,3-triazole- and 1,2,4-triazole-based compounds can be used as fluorescent probes. The compounds can optionally be substituted with one or two fluorine atoms. The compounds are effective as specific endoplasmatic reticulum (ER)-trackers for live cell imaging in a fungal cell, such as a pathogenic yeast. Compositions including the fluorescent probes can be used in methods for tracking the endoplasmic reticulum in a fungal cell.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

The present application claims the benefit of U.S. Provisional Application No. 62/723,707, filed Aug. 28, 2018, which is hereby incorporated herein by reference in its entirety.

### BACKGROUND OF THE INVENTION

### Field of the Invention

The present invention relates to particular 1,2,3-triazole- and 1,2,4-triazole-based fluorescent probes, which are effective and specific endoplasmatic reticulum-trackers for live cell imaging of pathogenic yeast, compositions thereof, and method of use.

Abbreviations: DCM, dichloromethane; DIPEA, N,N-diisopropylethylamine; DMF, N,N′ -dimethylformamide; DMSO, dimethyl sulfoxide; ER, endoplasmic reticulum; FLC, fluconazole; HATU, hexafluorophosphate azabenzotriazole tetramethyl uranium; HBSS, Hank's balanced salt solution; HRESI, high-resolution electrospray ionization; ITR, itraconazole; MeOH, methanol; MIC, minimal inhibitory concentration; PBS, phosphate-buffered saline; THF, tetrahydrofuran; TLC, thin layer chromatography; YPD, yeast extract peptone dextrose.

### Description of the Related Art

Epifluorescence and confocal microscopy in combination with organelle-specific fluorescent dyes, also termed organelle trackers, are amongst the most common and broadly applied tools for studying biological processes in living cells (Xu et al., 2016). The cytoplasm of eukaryotic cells is embedded with organelles that have different essential roles in cellular functions. Organelle-specific dyes can be used as counterstains to help identifying the location of a target protein that is fluorescently labeled by a reporter fluorescent protein or a specific fluorescent antibody (Zhang et al., 2002). These imaging tools are also essential for studying the functions and dynamics of the different organelles during the cell cycle, as well as inter-organelle interactions and organization, and for evaluating the effects of small molecules (drugs, nutrients, and metabolites) on the assembly and functions of specific organelles.

During the recent decades, numerous fluorescent organelle trackers have been developed for use in biological and medical research as well as for diagnostic applications (Fernández-Suárez and Ting, 2008). The choice of a fluorescent tracker is based on spectral properties such as excitation and emission wavelengths, stability to bleaching, cell toxicity, and specificity for the organelle of interest. Notably, the outstanding diversity of eukaryotic cells in nature is manifested in structural and functional differences that can greatly affect the performance of an organelle tracker. For example, the suitability of an organelle-specific tracker based on binding to a specific protein depends on the expression level of said protein and its subcellular distribution in the specific cell investigated. Other variable cellular characteristics that can perturb the specificity of an organelle tracker involve the expression of efflux pumps that reduce the concentration of the dye in the cell, cell permeability, and off-target binding sites (Xu et al., 2016). While most of the commercially available organelle trackers were developed and optimized for use in mammalian or plant cells, there are relatively few examples of trackers that specifically label organelles in fungal cells.

In the recent decades, there is an increase in the percentage of serious infections caused by drug-resistant pathogenic fungi. Members of the Candida genus such as Candida albicans and Candida glabrata, and the highly drug resistant Candida auris, are the most commonly encountered fungal pathogens that cause high mortality rates, especially amongst patients with a compromised immune system. Unfortunately, the urgent need to develop novel and more effective antifungal drugs is confounded by the very limited number of drug targets available in the fungal cell as a result of the evolutionary similarity between fungal and mammalian cells.

The predominant target for antifungal drug development is ergosterol, an essential sterol component of the fungal cell plasma membrane, and its biosynthetic pathway. Ergosterol has the same functions in the fungal membrane as cholesterol has in the plasma membrane of mammalian cells. Antifungal azoles that are used as first-line drug treatment for fungal infections inhibit the activity of CYP51, a cytochrome P450 lanosterol 14α-demethylase that is involved in the biosynthesis of ergosterol Like several other enzymes involved in ergosterol biosynthesis, CYP51 localizes primarily to the ER. As the ER is the main location of ergosterol biosynthesis, visual information about the assembly, shape-dynamics and components of the ER in cells of fungal pathogens would be of great value; however, no examples of ER-specific trackers optimal for use in pathogenic yeasts are currently available.

The dicarbocyanine dye DiOC₆ (3,3′-dihexyloxacarbocyanine iodide) was previously reported to stain the ER of cells of the baker's yeast Saccharomyces cerevisiae; however, this dye non-specifically labels intracellular membranes, and is also cytotoxic. Moreover, depending on the concentration and the specific strain, DiOC₆ was shown to label either the ER or the mitochondria of the yeast cell. Two other ER trackers that were developed for mammalian cells, a BODIPY-based brefeldin A derivative and the dapoxyl-based Blue-White DPX, were used for ER labeling in hyphal tips of the tree fungus Pisolithus tinctorius. Blue-White DPX was also used for ER labeling of Aspergillus oryzae, a filamentous fungus used for fermentation of soybeans, and of S. cerevisea.

### SUMMARY OF THE INVENTION

Recent publications suggest that the subcellular distribution of antifungal azoles such as fluconazole can be altered by attaching different fluorescent dyes to the pharmacophore structure thereof. As particularly shown, attachment of dansyl or Cy5 dyes results in antifungal azoles that localize mainly to the mitochondria of Candida yeast cells during the first few hours of exposure (Benhamou et al., 2017; Kim et al., 2018). On the other hand, it has now been found that attachment of a 7-(diethyl)-aminocoumarin dye to the pharmacophore of fluconazole directed the drug mainly to the ER. Compared to the mitochondria-localized azoles, the ER-localized azole shown displayed up to two orders of magnitude improvement in antifungal activity and reduced the growth of drug-tolerant fungal subpopulations in a panel of Candida (Benhamou et al., 2018).

As found in accordance with the present invention, fluorescent probes mimicking the pharmacophore of the antifungal drugs fluconazole and itraconazole, and based on the fluorescent dyes boron-dipyrromethene (BODIPY) or coumarin, linked to particular 1,2,3-triazole or 1,2,4-triazole, are both effective and specific ER trackers, as shown with a panel of fungal pathogens from the Candida genus, and display several superior properties compared to commercially available ER trackers.

In one aspect, the present invention thus provides a fluorescent azole probe (also referred to as an “ER-tracker”) of the formula I:

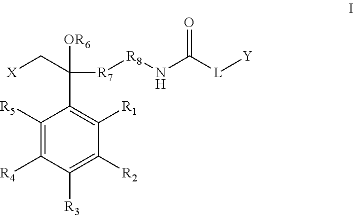

wherein:

X is selected from 1,2,3-triazole optionally substituted with one or two F atoms, or 1,2,4-triazole substituted with one or two F atoms, attached via a nitrogen atom thereof to the adjacent —CH₂—;

L is absent, or a linker selected from (C₁-C₆)alkylene, (C₂-C₆)alkenylene, or (C₂-C₆)alkynylene, and optionally interrupted with an aromatic or aliphatic ring;

Y is a fluorescent dye selected from BODIPY or coumarin, attached via a carbon atom thereof and optionally substituted with at least one substituent each independently selected from (C₁-C₆)alkyl, —O—(C₁-C₆)alkyl, Cl, Br, I, —CN, or —N(R′)₂ wherein R¹ each independently is H, (C₁-C₆)alkyl, (C₂-C₆)alkenyl, or (C₂-C₆)alkynyl, or the two R's together with the N atom to which they are attached form 5- or 6-membered heterocyclic ring optionally containing further 1 or 2 heteroatoms selected from N, O and S, provided that when Y is substituted with two (C₁-C₆)alkyl groups at two adjacent carbon atoms thereof, said (C₁-C₆)alkyl groups together with the carbon atoms to which they are attached may form a 5- or 6-membered carbocyclic ring;

R_{1,} R_{2,} R_{3,} R_{4,} and R₅ each independently is selected from H, halogen, —CN, or a heteroaryl containing N atom and optionally at least one further heteroatom selected from N, O and S;

R₆ is H or —CH₂-;

R₇ is —CH₂— or —O—CH₂-CH₂—O—; and

R₈ is absent, arylene-diyl, or (C₁-C₆)alkylene,

provided that when R₆ is H, R₇ is —CH₂—; and when R₆ is —CH₂—, R₇ is —O—CH₂—CH₂—O—, and R₆ together with one of the carbon atoms of R₇ form a 5- or 6-membered heterocyclic ring.

The ER-tracker specifically shown herein are illustrated in Table 1 and identified by the Arabic numbers 1-6, and the procedures for the preparation thereof are shown in the Appendix hereinafter, Schemes 1-3.

As shown herein, whereas the commercial ER-tracker BODIPY TR Glibenclamide did not stain any of the strains in the tested Candida panel, and the commercial ER-tracker Blue-White DPX stained Candida glabrata cells only, the fluorescent azole probes, also referred to as “ER-trackers”, of the present invention stained the ER of all of the strains in the panel both effectively and specifically, with less background signal. In fact, while non-specific labeling of additional intracellular compartments, such as lipid droplets, was observed in cells stained with Blue-White DPX and DiOC_{6,} no such non-specific labeling was observed in cells stained with any of the probes disclosed herein. As further shown, whereas DiOC₆ is known to cause cytotoxic effects at concentrations used for ER labeling, fluorescent azole probes of the present invention, which contain 1,2,3-triazole ring, displayed no antifungal activity at concentrations significantly higher than those used for ER labeling, and are therefore suitable for live cell imaging experiments.

Moreover, in a simultaneous labeling of both the ER and the mitochondria of Candida albicans cells, using a combination of one of the probes of the present invention and a mitochondrial tracker, it was possible to identify inter-organelle organization and colocalization sites that can be attributed to membrane contact sites between the two organelles in live Candida cells. The ER-trackers disclosed herein thus offer robust new molecular tools for the study of the ER and its interconnections with other organelles in live cells of important fungal pathogens.

In another aspect, the present invention provides a composition comprising an ER-tracker of the formula I as defined above.

In a further aspect, the present invention provides a method for tracking the ER in a fungal cell, said method comprising incubating said fungal cell with a compound of the formula I as defined above, or a composition comprising said compound; irradiating said fungal cell with a light at a wavelength within the excitation spectrum of said compound; and imaging the light emitted from said compound.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows localization of probes 2,3,5 and 6 and commercial ER trackers in C. albicans SN152. Cells were incubated for 60 min with Blue-White DPX (red, 1 μM, panels A-B), DiOC₆ (yellow, 1 μg/mL, panels C-D), probe 2 (cyan, 10 μM, panels E-F), probe 3 (cyan, 10 μM, panels G-H), probe 5 (green, 10 μM, panels I-J), or probe 6 (green, 10 μM, panels K-L). For each dye, the brightfield image is shown on the left and the fluorescent image on the right. The bandpass filters used for imaging were ex: 390 nm and em: 525 nm (Blue-White DPX); ex: 440 nm and em: 480 nm (DiOC_{6,} probes 2-3); and ex: 470 nm and em: 525 nm (probes 5-6). Scale bars, 5 μm.

FIG. 2 shows images of yeast cells of C. glabrata strain 2001 that were incubated with Blue-White DPX (red, 1 μM) and probe 5 (green, 10 μM) for 60 min (panels A-D); images of yeast cells of C. albicans expressing Eno1-mCherry that localizes mainly to the nucleus (red), that were incubated with probe 5 (green, 10 μM) for 60 min (panels E-H); images of yeast cells of C. albicans SN152 that were incubated with probe 5 (green, 10 μM) for 60 min (panels I-J); and images of yeast cells of C. albicans erg11ΔΔ/erg3ΔΔ that were incubated with probe 5 (green, 10 μM) for 60 min (panels K-L). Scale bars, 5 μm.

FIG. 3 shows MCSs between the ER and the mitochondria visualized by fluorescence microscopy in live C. albicans SN152 cells. Representative images of Cy5-based mitochondrial dye (red) after excitation at 560/25 nm (emission was monitored at 684/24 nm) (panel A). Representative images of probe 3 (cyan) after excitation at 427/10 nm (emission was monitored at 510/20 nm) (panel B). Merged images (arrowheads mark sites of co-localization) (panel C). Lower panels D-F are zoom-in of regions boxed in upper panels A-C, respectively. Cells were incubated for 5 min with 1 μg/mL Cy5-based mitochondrial dye and 10 μM of probe 3. Images were processed by convolution with a Gaussian function for smoothing using ImageJ program.

FIGS. 4A-4F show absorption and emission spectra of probes 1-6 (10 μM in PBS, pH=7.4), respectively. Emission spectrum was measured after excitation at 420 nm.

FIG. 5 shows localization of azole probes and commercial ER-trackers in C. glabrata 2001. Cells were incubated for 60 min with probe 1 (cyan, 10 μM, panels A-B); probe 2 (cyan, 10 μM, panels C-D); probe 3 (cyan, 10 μM, panels E-F); DiOC₆ (yellow, 1 μg/mL, panels G-H); probe 4 (green, 10 μM, panels I-J); probe 5 (green, 10 μM, panels K-L); probe 6 (green, 10 μM, panels M-N); or Blue-White DPX (red, 1 μM, panels O-P). The bandpass filters used for imaging were ex: 390 nm and em: 525 nm (Blue-White DPX); ex: 427 nm and em: 510 nm (DiOC_{6,} probes 1-3); and ex: 485 nm and em: 525 nm (probes 4-6). Scale bars, 5 μm.

FIG. 6 shows localization of azole probes and commercial ER-trackers in C. glabrata 66032. Cells were incubated for 60 min with probe 1 (cyan, 10 μM, panels A-B); probe 2 (cyan, 10 μM, panels C-D); probe 3 (cyan, 10 μM, panels E-F); DiOC₆ (yellow, 1 μg/mL, panels G-H); probe 4 (green, 10 μM, panels I-J); probe 5 (green, 10 μM, panels K-L); probe 6 (green, 10 μM, panels M-N); or Blue-White DPX (red, 1 μM, panels O-P). The bandpass filters used for imaging were were ex: 390 nm and em: 525 nm (Blue-White DPX); ex: 440 nm and em: 480 nm (DiOC_{6,} probes 1-3); and ex: 470 nm and em: 525 nm (probes 4-6). Scale bars, 5 μm.

FIG. 7 shows localization of azole probes and commercial ER-trackers in C. albicans 24433. Cells were incubated for 60 min with probe 1 (cyan, 10 μM, panels A-B); probe 2 (cyan, 10 μM, panels C-D); probe 3 (cyan, 10 μM, panels E-F); DiOC₆ (yellow, 1 μg/mL, panels G-H); probe 4 (green, 10 μM, panels I-J); probe 5 (green, 10 μM, panels K-L); probe 6 (green, 10 μM, panels M-N); or Blue-White DPX (red, 1 μM, panels O-P). The bandpass filters used for imaging were ex: 390 nm and em: 525 nm (Blue-White DPX); ex: 440 nm and em: 480 nm (DiOC_{6,} probes 1-3); and ex: 470 nm and em: 525 nm (probes 4-6). Scale bars, 5 μm.

FIG. 8 shows localization of probes 1 and 4 in C. albicans SN152. Cells were incubated for 60 min with probe 1 (cyan, 10 Mm, panels A-B); or probe 4 (green, 10 μM, panels C-D). The bandpass filters used for imaging were ex: 440 nm and em: 480 nm (probe 1); and ex: 470 nm and em: 525 nm (probe 4). Scale bars, 5 μm.

FIG. 9 shows localization of azole probes in C. albicans Eno 1-mCherry. Cells were incubated for 60 min with probe 1 (cyan, 10 μM, panels A-D); probe 2 (cyan, 10 μM, panels E-H); probe 3 (cyan, 10 μM, panels I-L); probe 4 (green, 10 μM, panels M-P); probe 5 (green, 10 μM, panels Q-T); or probe 6 (green, 10 μM, panels U-X). The bandpass filters used for imaging were ex: 440 nm and em: 480 nm (probes 1-3); ex: 470 nm and em: 525 nm (probes 4-6); and ex: 585 nm and em: 630 nm (Eno1 mCherry). Scale bars, 5 μm.

FIG. 10 shows Candida cells treated with BODIPY™ TR Glibenclamide. Cells were incubated for 60 min with BODIPY™ TR Glibenclamide (red, 1 μM) in C.albicans 24433 (panels A-B); and BODIPY™ TR Glibenclamide (red, 1 μM) in C.glabrata 66032 (panels C-D). The bandpass filters used for imaging BODIPY™ TR Glibenclamide were ex: 585 nm and em: 630 nm. Scale bars, 5 μm.

FIG. 11 shows Candida cells treated with 7-(diethyl)-aminocoumarin ethyl ester. Cells were incubated for 60 min with coumarin ethyl ester (cyan, 10 μM) in C.albicans 24433 (panels A-B); and coumarin ethyl ester (cyan, 10 μM) in C.glabrata 2001 (panels C-D). The bandpass filters used for imaging coumarin ethyl ester were ex: 440 nm and em: 480 nm; Scale bars, 5 μm.

FIG. 12 shows Candida cells treated with BODIPY methyl ester. Cells were incubated for 60 min with BODIPY methyl ester (green, 10 μM) in C.albicans 24433 (panels A-B); and BODIPY methyl ester (green, 10 μM) in C.glabrata 2001 (panels C-D). The bandpass filters used for imaging azoles BODIPY methyl ester were ex: 470 nm and em: 525 nm; Scale bars, 5 μm.

FIG. 13 shows localization of azoles probes in C. albicans erg11ΔΔ/erg3ΔΔ. Cells were incubated for 60 min with probe 1 (cyan, 10 μM, panels A-B); probe 2 (cyan, 10 μM, panels C-D); probe 3 (cyan, 10 μM, panels E-F); probe 4 (green, 10 μM, panels G-H); probe 5 (green, 10 μM, panels I-J); and probe 6 (green, 10 μM, panels K-L). The bandpass filters used for imaging were ex: 440 nm and em: 480 nm (probes 1-3); and ex: 470 nm and em: 525 nm (probes 4-6). Scale bars, 5 μm.

### DETAILED DESCRIPTION OF THE PREFERRED EMBODIMENT

In one aspect, the present invention provides an ER-tracker of the formula I as defined above.

The term “(C₁-C₆)alkyl” as used herein typically means a linear or branched saturated hydrocarbon radical having 1-6 carbon atoms and includes, e.g., methyl, ethyl, n-propyl, isopropyl, n-butyl, sec-butyl, isobutyl, tert-butyl, 2-methylpropyl, n-pentyl, isopentyl, neopentyl, 2-methylbutyl, 1,1-dimethylpropyl, 2,2-dimethylpropyl, n-hexyl, isohexyl, 2-methylpentyl, 3-methylpentyl, 1,1-dimethylbutyl, 2,2-dimethylbutyl, 2-ethylbutyl, and the like, wherein preferred are (C₁-C₄)alkyl or (C₁-C₃)alkyl groups such as methyl, ethyl, n-propyl, isopropyl, n-butyl, or tert-butyl.

The terms “(C₂-C₆)alkenyl” and “(C₂-C₆)alkynyl” typically mean linear or branched hydrocarbon radicals having 2-6 carbon atoms and one or more, e.g., one, two, or three, double or triple bonds, respectively.

The term “(C₁-C₆)alkylene” typically means a divalent linear or branched hydrocarbon radical having 1-6 carbon atoms and includes, e.g., methylene, ethylene, propylene, butylene, 2-methylpropylene, pentylene, 2-methylbutylene, hexylene, 2-methylpentylene, 3-methylpentylene, 2,3-dimethylbutylene, and the like. Preferred are (C₁-C₄)alkylenes, e.g., (C₁-C₃)alkylenes such as methylene, ethylene, and propylene.

The terms “(C₂-C₆)alkenylene” and “(C₂-C₆)alkynylene” refer to linear or branched divalent hydrocarbon radicals having 2-6 carbon atoms, and one or more double or triple bonds, respectively.

The term “halogen” as used herein refers to a halogen and includes fluoro, chloro, bromo, and iodo, but it is preferably fluoro or chloro.

The term “aliphatic ring” or “carbocyclic ring” as used herein interchangeably refers to cyclic hydrocarbon having 3-8 carbon atoms, which may be saturated or unsaturated, i.e., containing at least one unsaturated bond. Examples of saturated carbocyclic rings include, without limiting, cyclopropane, cyclobutane, cyclopentane, cyclohexane, cycloheptane, and cyclooctane; and examples of unsaturated carbocyclic rings include, without being limited to, cyclopropene, cyclobutene, cyclopentene, cyclohexene, cycloheptene, and cyclooctene. Preferred are 5-7 membered carbocyclic rings.

The term “heterocyclic ring” as used herein denotes a mono- or poly-cyclic non-aromatic ring containing at least one carbon atom and one or more heteroatoms each independently selected from nitrogen, oxygen, and sulfur, which may be saturated or unsaturated, i.e., containing at least one unsaturated bond. Preferred are 5- or 6-membered heterocyclic rings. Non-limiting examples of heterocyclic rings include pyrrolidine, piperidine, pyridine, dihydropyridine, tetrahydropyridine, pyrazole, pyrazoline, pyrazolidine, piperazine, imidazolidine, imidazoline, tetrahydropyrimidine, dihydrotriazine, azepane, morpholine, oxazolidine, oxazole, oxadiazole, oxazoline, dihydrooxadiazole, thiomorpholine, thiazolidine, thiazole, thiadiazole, thiazoline, dioxole, dioxolane, pyran, dihydropyran, tetrahydropyran, furan, tetrahydrofuran, and the like.

The term “aromatic ring” as used herein denotes an optionally substituted aromatic carbocyclic group having 6-10 carbon atoms consisting of a single ring or condensed multiple rings such as, but not limited to, phenyl and naphthyl. The term “aryl” denotes a radical derived from an aromatic ring as defined herein by removal of a hydrogen atom from any of the ring atoms. The term “arylene-diyl” refers to a divalent radical derived from an aromatic ring as defined herein by removal of two hydrogen atoms from any of the ring atoms, e.g., phenylene and naphthylene.

The term “heteroaromatic ring” as used herein denotes a fully unsaturated mono- or poly-cyclic aromatic ring in which at least one atom forming the ring backbone is a heteroatom selected from nitrogen, oxygen, and sulfur. Preferred are 5- or 6-membered heteroaromatic rings. The term “heteroaryl” denotes a radical derived from an aromatic ring as defined herein by removal of a hydrogen atom from any of the ring atoms. Examples of heteroaryl radicals include, without being limited to, monocyclic heteroaryl radicals such as pyrrolyl, furyl, thienyl, thiazinyl, pyrazolyl, pyrazinyl, imidazolyl, oxazolyl, isoxazolyl, thiazolyl, isothiazolyl, pyridyl, pyrimidinyl, 1,2,3-triazinyl, 1,3,4-triazinyl, and 1,3,5-triazinyl; and polycyclic heteroaryl radicals such as benzofuranyl, isobenzofuranyl, indolyl, quinolinyl, isoquinolinyl, imidazo[1,2-α]pyridyl, benzimidazolyl, benzthiazolyl, benzoxazolyl, pyrido[1,2-a]pyrimidinyl and 1,3-benzodioxinyl.

In certain embodiments, the present invention provides a compound of the formula I, wherein X is 1,2,3-triazole optionally substituted with one or two F atoms. In particular such embodiments, X is 1,2,3-triazole.

In other embodiments, the present invention provides a compound of the formula I, wherein X is 1,2,4-triazole substituted with one or two F atoms.

In certain embodiments, the present invention provides a compound of the formula I, wherein R_{1,} R_{2,} R_{3,} R_{4,} R₅ each independently is selected from H, F, or Cl. In some particular such embodiments, one of R_{1,} R_{2,} R_{3,} R_{4,} and R₅ is F or Cl, and the others of R_{1,} R_{2,} R_{3,} R_{4,} and R₅ each is H. In other particular such embodiments, two of R_{1,} R_{2,} R_{3,} R₄ and R_{5,} i.e., R₁ and R₂, R₁ and R_{3,} R₁ and R_{4,} R₁ and R_{5,} R₂ and R₃, R₂ and R_{4,} R₂ and R_{5,} R₃ and R_{4,} R₃ and R_{5,} or R₄ and R_{5,} each is F or Cl, and the others of R_{1,} R_{2,} R_{3,} R_{4,} and R₅ each is H. More particular such embodiments are those wherein either R₁ and R_{3,} or R₃ and R_{5,} but preferably R₁ and R_{3,} each is F or Cl; and R_{2,} R_{4,} and the other one of R₁ and R₅ each is H.

In certain embodiments, the present invention provides a compound of the formula I, wherein R₆ is H; and R₇ is —CH₂—. In particular such embodiments, R₈ is absent, or (C₁-C₆)alkylene, e.g., (C₁-C₃)alkylene. More particular such embodiments are those wherein R₈ is absent.

In certain embodiments, the present invention provides a compound of the formula I, wherein R₆ is —CH₂—; R₇ is —O—CH₂—CH₂—O—; and R₆ together with one of the carbon atoms of R₇ form a 5- or 6-membered, preferably 5-membered, heterocyclic ring. In particular such embodiments, R₈ is (C₁-C₆)alkylene, e.g., (C₁-C₃)alkylene, or arylene-diyl, e.g., phenylene.

In certain embodiments, the present invention provides a compound of the formula I, wherein L is absent, or (C₁-C₆)alkylene, e.g., methylene, ethylene, propylene, or butylene, optionally interrupted with an aromatic or aliphatic ring.

In certain embodiments, the present invention provides a compound of the formula I, wherein Y is coumarin (i.e., 2H-chromen-2-one), either non-substituted or substituted with at least one substituent each independently selected from (C₁-C₆)alkyl, e.g., methyl, ethyl, n-propyl, or isopropyl, —O—(C₁-C₆)alkyl, e.g., —O-methyl, —O-ethyl, —O-propyl, or —O-isopropyl, Cl, Br, I, or —N(R′)_{2,} wherein R′ each independently is H, (C₁-C₆)alkyl, e.g., (C₁-C₃)alkyl, or the two R′s together with the N atom to which they are attached form an optionally substituted 5- or 6-membered heterocyclic ring optionally containing further 1 or 2 heteroatoms selected from N, O and S. In some of these embodiments, the coumarin is substituted with two (C₁-C₆)alkyl groups at two adjacent carbon atoms thereof, and said (C₁-C₆)alkyl groups together with the carbon atoms to which they are attached form a 5- or 6-membered carbocyclic ring. In particular such embodiments, Y is coumarin substituted at any position thereof, with —N(R′)_{2,} wherein R′ each independently is H or (C₁-C₆)alkyl. More particular such embodiments are hose wherein R′ each is (C₁-C₃)alkyl, i.e., methyl, ethyl, n-propyl, or isopropyl. In specific such embodiments, Y is diethylaminocoumarin, e.g., 7-diethylaminocoumarin such as 7-(diethylamino)-coumarin-3-yl exemplified herein.

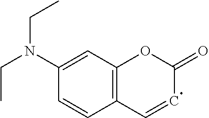

In certain embodiments, the present invention provides a compound of the formula I, wherein Y is BODIPY (i.e., 4,4-difluoro-4-bora-3a,4a-diaza-s-indacene), either non-substituted or substituted with at least one substituent each independently selected from (C₁-C₆)alkyl, e.g., methyl, ethyl, n-propyl, or isopropyl, —O—(C₁-C₆)alkyl, e.g., —O-methyl, —O-ethyl, —O-propyl, or —O-isopropyl, Cl, Br, I, or —N(R′)_{2,} wherein R′ each independently is H, (C₁-C₆)alkyl, e.g., (C₁-C₃)alkyl, or the two R′s together with the N atom to which they are attached form an optionally substituted 5- or 6-membered heterocyclic ring optionally containing further 1 or 2 heteroatoms selected from N, O and S. In some of these embodiments, the BODIPY is substituted with two (C₁-C₆)alkyl groups at two adjacent carbon atoms thereof, and said (C₁-C₆)alkyl groups together with the carbon atoms to which they are attached form a 5- or 6-membered carbocyclic ring. Specific examples of BODIPY derivatives are disclosed, e.g., in Loudet and Burgess (2007). In particular such embodiments, Y is BODIPY substituted at any two positions thereof with two identical or different (C₁-C₃)alkyl groups. More particular such embodiments are those wherein said BODIPY is substituted with two identical (C₁-C₃)alkyl groups, e.g., with two methyl groups. In specific such embodiments, Y is 4,4-difluoro-1,4-dimethyl-4-bora-3a,4a-diaza-s-indacen, e.g., 4,4-difluoro-1,4-dimethyl- 4-bora-3 a,4a-diaza-s-indacen-5-yl exemplified herein.

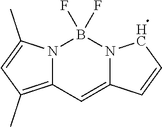

In certain embodiments, the present invention provides a compound of the formula I, wherein X is 1,2,3-triazole optionally substituted with one or two F atoms, or 1,2,4-triazole substituted with one or two F atoms, preferably 1,2,3-triazole; R_{1,} R_{2,} R_{3,} R_{4,} R₅ each independently is selected from H, F, or Cl; L is absent, or (C₁-C₆)alkylene optionally interrupted with an aromatic or aliphatic ring; and Y is coumarin substituted with —N(R′)_{2,} wherein R′ each independently is H, or (C₁-C₆)alkyl, preferably (C₁-C₃)alkyl, i.e., methyl, ethyl, n-propyl, or isopropyl; or BODIPY substituted with two identical or different (C₁-C₃)alkyl groups, i.e., methyl, ethyl, n-propyl, or isopropyl, preferably with two identical (C₁-C₃)alkyl groups, e.g., two methyl groups. In particular such embodiments, two of R₁, R_{2,} R_{3,} R₄ and R_{5,} i.e., R₁ and R₂, R₁ and R_{3,} R₁ and R_{4,} R₁ and R_{5,} R₂ and R_{3,} R₂ and R_{4,} R₂ and R_{5,} R₃ and R_{4,} R₃ and R_{5,} or R₄ and R_{5,} each is F or Cl, and the others of R₁, R_{2,} R_{3,} R_{4,} and R₅ each is H; L is absent, or (C₁-C₄)alkylene; and Y is 7-diethylaminocoumarin such as 7-(diethylamino)-coumarin-3-yl; or 4,4-difluoro-1,4-dimethyl-4-bora-3a,4a-diaza-s-indacen-5-yl. More particular such embodiments are those wherein either R₁ and R_{3,} or R₃ and R_{5,} but preferably R₁ and R_{3,} each is F or Cl; and R_{2,} R_{4,} and the other one of R₁ and R₅ each is H.

In certain particular embodiments as disclosed hereinabove, R₆ is H; and R₇ is —CH₂—. More particular such compounds are those wherein R₈ is absent. Specific such ER trackers are those wherein (i) X is 1,2,3-triazole; R₁ and R₃ each is F or Cl; R_{2,} R_{4,} and R₅ each is H; R₆ is H; R₇ is —CH₂—; R₈ is absent; L is absent; and Y is 7-(diethylamino)-coumarin-3-yl, herein identified compound 2 and 3, respectively; or (ii) X is 1,2,3-triazole; R₁ and R₃ each is F or Cl; R_{2,} R_{4,} and R₅ each is H; R₆ is H; R₇ is —CH₂—; R₈ is absent; L is —(CH₂)₂—; and Y is 4,4-difluoro-1,4-dimethyl-4-bora-3a,4a-diaza-s-indacen-5-yl, herein identified compound 5 or 6, respectively (Table 1).

TABLE 1
Compounds 1-6 disclosed herein

| | |
|---|---|
| 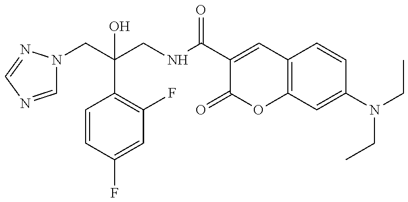 [SMILES: CCN(CC)c1ccc2cc(C(=O)NCC(O)(Cn3cncn3)c3ccc(F)cc3F)c(=O)oc2c1] | 1 |
| 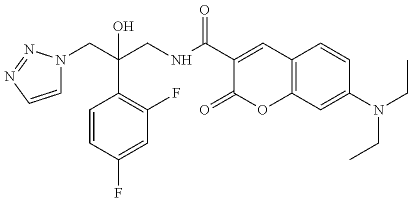 [SMILES: CCN(CC)c1ccc2cc(C(=O)NCC(O)(Cn3ccnn3)c3ccc(F)cc3F)c(=O)oc2c1] | 2 |
| 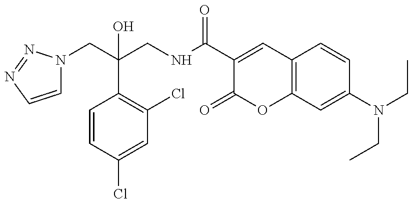 [SMILES: CCN(CC)c1ccc2cc(C(=O)NCC(O)(Cn3ccnn3)c3ccc(Cl)cc3Cl)c(=O)oc2c1] | 3 |
| 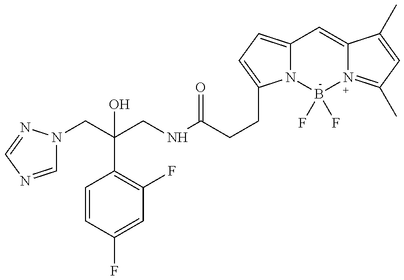 | 4 |
| 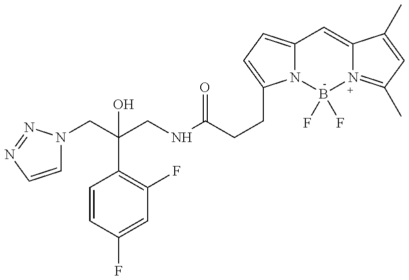 | 5 |
| 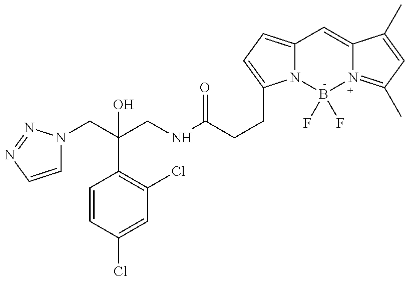 | 6 |

In other particular embodiments as disclosed hereinabove, R₆ is —CH₂—; R₇ is —O—CH₂—CH₂—O—; and R₆ together with one of the carbon atoms of R₇ form a 5-membered heterocyclic ring. More particular such embodiments are those wherein R₈ is absent or arylene-diyl such as phenylene.

As found and shown in detail in the Experimental section herein, the compounds of the formula I, further referred to herein as “fluorescent azoles” or “azole-based fluorescent probes” have no antifungal activity and are highly effective and specific in tracking the ER in live pathogenic fungal cells, displaying several superior properties compared to the ER trackers currently known.

In another aspect, the present invention thus provides a composition comprising an azole-based fluorescent probes as disclosed herein, i.e., a compound of the formula I as defined in any one of the embodiments above. Particular such compositions are those wherein said compound is based on 1,2,3-triazole optionally substituted with one or two F atoms, e.g., the 1,2,3-triazole-based fluorescent probe herein identified probes 2, 3, 5, or 6. Other particular such compositions are those wherein said compound is based on 1,2,4-triazole substituted with one or two F atoms.

In a further aspect, the present invention relates to a method for tracking the ER in a fungal cell, more specifically a live pathogenic fungal cell, said method comprising incubating said fungal cell with an azole-based fluorescent probe as disclosed herein, i.e., a compound of the formula I as defined in any one of the embodiments above, or a composition comprising said azole-based fluorescent probe; irradiating said fungal cell with a light at a wavelength within the excitation spectrum of said compound; and imaging the light emitted from said compound. In certain embodiments, the compound incubated with said fungal cell is based on 1,2,3-triazole optionally substituted with one or two F atoms, e.g., the 1,2,3-triazole-based fluorescent probe herein identified probes 2, 3, 5, or 6. In other embodiments, said compound is based on 1,2,4-triazole substituted with one or two F atoms.

The invention will now be illustrated by the following non-limiting Examples.

### EXAMPLES

### Experimental

General chemistry methods and instrumentation. ¹H-NMR spectra (including 1D-TOCSY) were recorded on BrukerAvance™ 400 or 500 MHz spectrometers, and chemical shifts (reported in ppm) were calibrated to CDCl₃ (d=7.26) when CDCl₃ was the solvent, or to CD₃OD (d=3.31) when CD₃OD was the solvent. ¹³C-NMR spectra were recorded on BrukerAvance™ 400 or 500 MHz spectrometers at 100 or 125 MHz. ¹⁹F-NMR spectra were recorded on BrukerAvance™ 400 or 500 MHz spectrometers at 375 or 470 MHz. Multiplicities are reported using the following abbreviations: b=broad, s=singlet, d=doublet, t=triplet, q=quartet. Coupling constants (J) are given in Hertz. HRESI mass spectra were measured on a Waters Synapt instrument. Chemical reactions were monitored by TLC (Merck, Silica gel 60 F254). Visualization was achieved using a cerium molybdate stain (5 g (NH₄)₂Ce(NO₃)_{6,} 120 g (NH₄)₆Mo₇O₂₄·4H₂O, 80 mL H₂SO₄, 720 mL H₂O) or with UV lamp. Unless otherwise stated, all chemicals were obtained from commercial sources. Compounds were purified using Geduran® Si 60 chromatography (Merck). The SpectraMax i3x Platform spectrophotometer from Molecular Devices was used for the measurement of the excitation and absorbance spectra of the fluorescent probes.

Synthesis of 1,2,3-azole-2,4-dichloro synthon (1-amino-2-(2,4-dichlorophenyl)-3-(1H-1,2,3-triazol-1-yl)propan-2-ol, 10)

Compound 7. As depicted in Scheme 1, step a, a mixture of 2-chloro-2′,4′-dichloroacetophenone (2 g, 9.0 mmol), 1,2,3-triazole (0.62 mL, 10.8 mmol), and sodium bicarbonate (0.9 g, 10.8 mmol) in toluene (25 mL) was refluxed for 4 h. The reaction was monitored by TLC (petroleum ether/ethyl acetate, 1:1). Upon completion, the reaction mixture was poured into crushed ice and extracted with toluene (2×25 mL). The combined organic layer was washed with H₂O (2×10 mL) and brine (20 mL), dried over MgSO₄, and concentrated to give the crude product as a brown oil. The product was then isolated by column chromatography on SiO₂ using petroleum ether/ethyl acetate (30:70) as eluent to afford compound 7 (0.9 g, 39%). ¹H NMR (400 MHz, CDCl₃) δ 7.80 (d, J=0.9 Hz, H-1, 1H), 7.78 (s, H-2, 1H), 7.70 (d, J=8.4 Hz, H-3, 1H), 7.53 (d, J=1.9 Hz, H-5, 1H), 7.41 (dd, J=8.4, 2.0 Hz, H-4, 1H), 5.90 (s, H-6, 2H). ¹³C NMR (100 MHz, CDCl₃) δ 191.49, 139.39, 133.90, 133.10, 132.82, 131.40, 130.78, 127.78, 125.43, 57.91.

Compound 8. As depicted in Scheme 1, step b, to a solution of 7 (0.7 g, 1.9 mmol) in toluene (10 ml) was added trimethylsulfoxonium iodide (0.71 g, 2.3 mmol) followed by the addition of 20% sodium hydroxide solution (0.1 ml). The reaction mixture was then heated at 60° C. for 4 h. Upon completion, the mixture was diluted with toluene (20 ml) and poured into chilled water. The organic layer was washed with H₂O (2×20 ml) and brine (20 ml), dried over MgSO_{4,} and concentrated to give the crude product as light brown oil. The product was then isolated by column chromatography on SiO₂ using petroleum ether/ethyl acetate (40:60) as eluent to afford compound 8 (0.69 g, 94%). ¹H NMR (400 MHz, CDCl₃) δ 7.70 (s, H-1, H-2, 2H), 7.42 (d, J=2.0 Hz, H-5, 1H), 7.17 (dd, J=8.3, 2.0 Hz, H-4, 1H), 7.09 (d, J=8.3 Hz, H-3, 1H), 5.16 (d, J=14.8 Hz, H-6, 1H), 4.69 (d, J=14.8 Hz, H-6, 1H), 3.00 (d, J=4.5 Hz, H-7, 1H), 2.93 (d, J=4.5 Hz, H-7, 1H). ¹³C NMR (100 MHz, CDCl₃) δ 135.13, 134.65, 133.23, 131.25, 130.63, 130.29, 128.93, 127.09, 58.61, 58.04, 52.01.

Compound 9. As depicted in Scheme 1, step c, to a solution of 8 (0.6 g, 2.2 mmol) in DMF (5 ml) was added sodium azide (0.29 g, 4.4 mmol). The reaction mixture was then heated at 65° C. for 12 hours. The reaction was monitored by TLC (petroleum ether/ethyl acetate, 3:7). Upon completion, the product was extracted with ethyl acetate, washed with H₂O (3×20 mL), dried over MgSO₄ and concentrated to give the crude product. The product was then isolated by column chromatography on SiO₂ using petroleum ether/ethyl acetate (50:50) as eluent to afford compound 9 as yellowish oil (0.69 g, 89%). ¹H NMR (400 MHz, CD₃OD) δ 7.82 (s, H-1, 1H), 7.59-7.56 (m, H-2, H-3, 2H), 7.44 (d, J=2.2 Hz, H-5, 1H), 7.21 (dd, J=8.7, 2.2 Hz, H-4, 1H), 5.26 (d, J=14.3 Hz, H-6, 1H), 4.89 (d, J=14.1 Hz, H-6, 1H), 4.15 (d, J=13.1 Hz, H-7, 1H), 3.81 (d, J=13.1 Hz, H-7, 1H). ¹³C NMR (100 MHz, CD₃OD) δ 134.40, 132.75, 130.72, 129.60, 129.18, 128.53, 125.22, 124.24, 74.93, 54.08, 52.55.

Compound 10. As depicted in Scheme 1, step d, to a solution of 9 (0.35 g, 1.1 mmol) in isopropanol (5 ml) was added 10% active palladium on carbon (0.035 g). The solution was stirred overnight at room temperature under a hydrogen atmosphere and filtered through celite. The filtrate was concentrated under reduced pressure. The product was isolated by column chromatography on SiO₂ using methanol/DCM (1:9) as eluent to afford compound 10 as transparent oil (0.13 g, 41%). ¹H NMR (400 MHz, CD₃OD) δ 7.81 (d, J=1.0 Hz, H-1, 1H), 7.57 (d, J=8.6 Hz, H-3, 1H), 7.55 (d, J=1.0 Hz, H-2, 1H), 7.45 (d, J=2.2 Hz, H-5, 1H), 7.20 (dd, J=8.6, 2.2 Hz, H-4, 1H), 5.28 (d, J=14.2 Hz, H-6, 1H), 4.79 (d, J=14.0 Hz, H-6, 1H), 3.53 (d, J=13.8 Hz, H-7, 1H), 3.12 (d, J=13.8 Hz, H-7, 1H). ¹³C NMR (100 MHz, CD₃OD) δ 136.84, 134.10, 132.30, 131.19, 130.28, 129.81, 126.77, 125.77, 76.47, 54.80, 46.21.

Synthesis of BODIPY fluorophore, 11

The BODIPY acid 11, used for preparing the azole fluorescent probes, was synthesized depicted in Scheme 2, following the procedure disclosed in Vanessa Saura et al. (2017).

Preparation of stock solutions of the tested probes. All fluorescent probes were dissolved in DMSO to final concentration of 5 mg/mL. The antifungal drugs FLC and ITR were purchased from Sigma Aldrich. FLC was dissolved in anhydrous ethanol, and ITR in DMSO to a final concentration of 5 mg/mL. ER-tracker DPX and DiOC₆ were purchased from Thermo Fisher, and BODIPY™ TR Glibenclamide was purchased from Setareh BioTech. BODIPY™ TR Glibenclamide and ER-tracker DPX were dissolved in DMSO to a final concentration of 1 mM. DiOC₆ was dissolved in DMSO to a final concentration of 1 mg/mL.

Minimal inhibitory concentration broth double-dilution assay. All strains were tested using the double-dilution method in 96-well plates (Corning). Yeast strains were grown in Casitone growth medium. Starter cultures were incubated for 24 h (37° C., 5% CO_{2,} aerobic conditions) and then diluted 1:100 into fresh medium. Compounds dissolved in ethanol or in DMSO were added to the casitone broth to form the mother liquor (32 μL stock solution in 1218 μL of casitone) at the starting concentration of 64 μg/mL. Next, 100 μL of serial double dilutions of compounds in Casitone (64, 32, 16, 8, 4, 2, 1, 0.5, 0.25, 0.125, 0.062, 0.031, 0.014, and 0.007 μg/mL) were prepared in flat-bottomed 96-well microplates (Corning). Control wells with no compounds, and wells without yeast cells containing each tested concentration of the compounds (blanks), were also prepared. An equal volume (100 μL) of yeast suspensions in Casitone broth that was prepared as follows: 900 mL doubly distilled H₂O, 9 g Casitone (bacto-casitone), 5 g yeast extract, 11.5 g sodium citrate dihydrate, 20 g glucose. The yeast suspension was added to each well for a final volume of 200 μL. The ethanol or the DMSO concentrations ranged from 0.0012% to 1.3%. The final inoculum was between 5×10⁴ colony-forming unit (CFU)/mL and 5×10⁵ CFU/mL; identical results were obtained when using 5×10³ CFU/mL as a final inoculum. After incubation for 24 h at 37° C. in 5% CO₂, MTT (3-[4,5-dimethylthiazole-2-yl]-2,5-diphenyltetrazolium bromide; 50 μL of a 1 mg/mL solution in H₂O) was added to each well followed by additional incubation at 37° C. for 2 h. Each concentration was tested in triplicate, and results were confirmed by two independent sets of experiments.

Growth conditions for live cell imaging. Cells were grown to log phase in liquid YPD overnight at 30° C. in a 10 ml tube. Cells were diluted 1:10 and then incubated for 2 h at 30° C. to log phase.

Cell staining. The fluorescent probes were added to a final concentration of 10 μM for probes 1-6 to cells from a log phase culture grown in YPD broth media at 30° C. Cells were then incubated in the dark for a total of 60 min. Before imaging, cells were washed once with PBS buffer. For ER-tracker dyes, DiOC₆ (1 μg/mL) and BODIPY™ TR Glibenclamide (1 μM), staining cells were incubated in a prepared Hanks' Balanced Salt Solution at 37° C. for 5 min and 30 min, respectively. For ER-tracker Blue-White DPX (1 μM), staining cells were incubated in YPD for 30 min at 30° C. For mitochondrial staining the previously reported Cy5-based mitochondrial dye was used (1 μg/mL). For staining, cells were incubated in YPD in the presence of dye for 5 min at 30° C. Cells were then washed with PBS buffer once.

Live cell imaging. Candida cells were resuspended in PBS buffer and 2 μL were placed on a glass slides and covered with glass coverslips. C. glabrata (ATCC 2001) cells treated with probes 1-6, all tested Candida cells treated with ER-tracker Blue-White DPX and inter-organelle interaction study were imaged on a MORE imaging system (TILL Photonics GmbH) with an Olympus UPlanApo 100X 1.3 NA oil immersion objective. The bandpass filter sets used to image probes 1-3 were excitation 427/10 nm and emission 510/20 nm. The bandpass filter sets used to image probes 4-6 were excitation 485/20 nm and emission 525/30 nm. The bandpass filter sets used to image ER-tracker Blue-White DPX were excitation 390/40 nm and emission 525/30 nm. The bandpass filter sets used to image Cy5-based mitochondrial dye were excitation 560/25 nm and emission 684/24 nm. C. albicans cells (ATCC 24433 and SN152) and C. glabrata (ATCC 66032) treated with probes 1-6 were imaged on a Nikon Ti microscope equipped with a Plan Apo VC 100X oil objective and a Zyla 5.5 sCMOS camera (Andor) run by NIS elements Ar software. The bandpass filter sets used to image probes 1-3, and DiOC₆-stained cells were excitation 440 nm and emission 480/40 nm. The bandpass filter sets used to image cells treated with probes 4-6, excitation 470 nm and emission 525/50 nm were used. For Eno1-mCherry, excitation 585 nm and emission 630/75 nm were used. To optimize the probes, the effects of the concentration (1, 5 and 10 μM), incubation time (5, 30, 60 and 120 min) and media (YPD and HBSS) were investigated. Images were processed using ImageJ program. The smoothed imaged were generated using the convolve filter (Normalize Kernel).

### Example 1

### Preparation of Azole Fluorescent Probes

Synthesis of the azole fluorescent probes was carried out by coupling of a fluorophore (7-diethylaminocoumarin or BODIPY) to an azole core (1,2,3-azole-2,4-dichloro synthon, 10, the corresponding difluoro compound, or the corresponding 1,2,4-azole-based compounds, as generally depicted in Scheme 3.

### Synthesis of Probes 1 and 2

Probes 1 and 2 were synthesized following the procedure previously reported (Benhamou et al., 2018).

### Synthesis of Probe 3

7-Diethylaminocoumarin-3-carboxylic acid (43 mg, 0.17 mmol) was dissolved in dry DMF (5 mL) under argon, treated with HATU (106 mg, 0.28 mmol) and triethylamine (0.08 mL, 0.56 mmol), then stirred for 10 min at 0° C. To the reaction mixture, 1-amino-2-(2,4-dichlorophenyl)-3-(1H-1,2,3-triazol-1-yl)propan-2-ol 10 (40 mg, 0.14 mmol) was added, and the solution was stirred at room temperature for 24 h. The reaction was monitored by TLC (petroleum ether/ethyl acetate, 3:7). Upon completion, the product was extracted with ethyl acetate and brine. The organic layers were washed with H₂O (3×20 mL), dried over MgSO_{4,} and concentrated to give the crude product. The product was isolated by column chromatography on SiO₂ using petroleum ether/ethyl acetate (30:70) as eluent to afford Probe 3 (69 mg, 93%) as a yellow powder. HRESI-MS m/z calculated for C₂₅H₂₆Cl₂N₅O₄, 530.1367; found for [M+H]⁺, 530.1366. ¹H NMR (500 MHz, CDCl₃) δ 9.18 (t, J=5.9 Hz, H-9, 1H), 8.52 (s, H-10, 1H), 7.80-7.75 (m, H-1, H-3, 2H), 7.62 (s, H-2, 1H), 7.40-7.34 (m, H-5, H-11, 2H), 7.14 (dd, J=8.6, 2.1 Hz, H-4, 1H), 6.81 (s, H-8, 1H), 6.63 (dd, J=9.0, 2.4 Hz, H-12, 1H), 6.44 (d, J=2.3 Hz, H-13, 1H), 5.09 (d, J=14.0 Hz, H-6, 1H), 4.83 (d, J=13.9 Hz, H-6, 1H), 4.31 (dd, J=14.7, 6.4 Hz, H-7, 1H), 3.80 (dd, J=14.7, 5.6 Hz, H-7, 1H), 3.44 (q, H-14, 4H), 1.22 (t, J=7.1 Hz, H-15, 6H). ¹³C NMR (125 MHz, CDCl₃) δ 166.74, 162.29, 157.90, 153.06, 148.44, 137.00, 134.67, 133.45, 131.39, 131.14, 130.96, 130.63, 127.46, 125.51, 110.20, 108.17, 96.59, 78.08, 55.57, 47.59, 45.17, 12.40.

### Synthesis of Probe 4

BODIPY acid 11, synthesized following the reported procedure (Vanessa Saura et al., 2017) (46 mg, 0.16 mmol), was dissolved in dry DMF (5 mL) under argon, treated with HATU (119 mg, 0.31 mmol) and triethylamine (0.08 mL, 0.62 mmol), then stirred for 10 min at 0° C. To the reaction mixture, 1-amino-2-(2,4difluorophenyl)-3-(1H-1,2,4-triazol-1-yl)propan-2-ol (40 mg, 0.16 mmol) synthesized following a reported procedure (Pore et al., 2015), was added and the solution was stirred at room temperature for 24 h. The reaction was monitored by TLC (MeOH/DCM, 1:9). Upon completion, the product was extracted with ethyl acetate, washed with H₂O (3×20 mL), dried over MgSO_{4,} and concentrated to give the crude product. The product was isolated by column chromatography on SiO₂ MeOH/DCM (5:95) as eluent to afford Probe 4 (54 mg, 65%) as a red powder. HRESI-MS m/z calculated for C₂₅H₂₅BF₄N₆O₂Na, 551.2002; found for [M+Na]⁺, 551.1956. ¹H NMR (400 MHz, CD₃OD) δ 8.30 (s, H-1, 1H), 7.76 (s, H-2, 1H), 7.43 (td, J=9.0, 6.6 Hz, H-3, 1H), 7.38 (s, H-14, 1H), 6.96-6.85 (m, H-13, H-4, 2H), 6.82 (ddd, J=8.2, 2.9, 1.3 Hz, H-5, 1H), 6.22-6.17 (m, H-12, H-15, 2H), 4.66 (d, J=14.3 Hz, H-6, 1H), 4.52 (d, J=14.3 Hz, H-6, 1H), 3.71 (s, H-7, 2H), 3.09 (t, J=7.5 Hz, H-11, 2H), 2.53 (t, J=7.5 Hz, H-10, 2H), 2.50 (s, H-16, 3H), 2.26 (s, H-17, 3H). ¹³C NMR (100 MHz, CD₃OD) 6 174.96, 164.13, 161.73, 160.47, 158.01, 156.73, 149.87, 144.70, 144.49, 135.15, 133.42, 131.06, 128.07, 124.32, 123.95, 119.99, 116.12, 110.69, 103.52, 75.30, 55.54, 34.07, 24.06, 13.42, 9.73. ¹⁹F NMR (375 MHz, CD₃OD) δ-109.42 (m, Fₚₐᵣₐ), −113.41 (m, Fₒᵣₜₕₒ), −146.36 (m, F_{BODIPY}).

### Synthesis of Probe 5

BODIPY acid 11, synthesized following the reported procedure (Vanessa Saura et al., 2017) (35 mg, 0.12 mmol), was dissolved in dry DMF (4 mL) under argon, treated with HATU (90 mg, 0.24 mmol) and triethylamine (0.07 mL, 0.47 mmol), then stirred for 10 min at 0° C. To the reaction mixture, 1-amino-2-(2,4difluorophenyl)-3-(1H-1,2,3-triazol-1-yl)propan-2-ol (30 mg, 0.12 mmol) synthesized following a reported procedure (Pore et al., 2015), was added and the solution was stirred at room temperature for 24 h. The reaction was monitored by TLC (MeOH/DCM, 5:95). Upon completion, the product was extracted with ethyl acetate, washed with H₂O (3×20 mL), dried over MgSO_{4,} and concentrated to give the crude product. The product was isolated by column chromatography on SiO₂ MeOH/DCM (2:98) as eluent to afford Probe 5 (33 mg, 53%) as a red powder. HRESI-MS m/z calculated for C₂₅H₂₅BF₄N₆O₂Na, 551.2014; found for [M+Na]⁺, 551.1975.¹H NMR (500 MHz, CD₃OD) δ 7.85 (d, J=1.0 Hz, H-1, 1H), 7.56 (d, J=0.8 Hz, H-2, 1H), 7.46-7.32 (m, H-3, H-14, 2H), 7.01-6.88 (m, H-13, H-4, 2H), 6.80 (td, J=8.3, 2.4 Hz, H-5, 1H), 6.29-6.15 (m, H-12, H-15, 2H), 4.89 (d, J=14.1 Hz, H-6, 1H), 4.68 (d, J=14.1 Hz, H-6, 1H), 3.73 (dd, J=14.5 Hz, H-7, 2H), 3.10 (t, J=7.5 Hz, H-11, 2H), 2.55 (t, J=7.5 Hz, H-10, 2H), 2.50 (s, H-16, 3H), 2.27 (s, H-17, 3H). ¹³C NMR (125 MHz, CD₃OD) 6 175.13, 163.91, 161.94, 160.26, 158.30, 156.69, 144.51, 135.16, 133.42, 132.32, 130.10, 128.09, 125.86, 124.35, 123.62, 119.99, 116.13, 110.64, 103.57, 75.52, 56.00, 34.06, 24.05, 13.43, 9.75. ¹⁹F NMR (470 MHz, CD₃OD) δ-109.25 (m, Fₚₐᵣₐ), −112.99 (m, Fₒᵣₜₕₒ), −146.02 (m, F_{BODIPY}).

### Synthesis of Probe 6

BODIPY acid 11, synthesized following the reported procedure (Vanessa Saura et al., 2017) (36 mg, 0.12 mmol), was dissolved in dry DMF (5 mL) under argon, treated with HATU (92 mg, 0.24 mmol) and triethylamine (0.07 mL, 0.47 mmol), then stirred for 10 min at 0° C. To the reaction mixture, 1-amino-2-(2,4difluorophenyl)-3-(1H-1,2,3-triazol-1-yl)propan-2-ol (35 mg, 0.12 mmol) synthesized following a reported procedure (Pore et al., 2015), was added and the solution was stirred at room temperature for 24 h. The reaction was monitored by TLC (MeOH/DCM, 5:95). Upon completion, the product was extracted with ethyl acetate, washed with H₂O (3×20 mL), dried over MgSO_{4,} and concentrated to give the crude product. The product was isolated by column chromatography on SiO₂ MeOH/DCM (2:98) as eluent to afford Probe 6 (60 mg, 79%) as a red powder. HRESI-MS m/z calculated for C₂₅H₂₅BCl₂F₂N₆O₂Na, 583.1411; found for [M+Na]⁺, 583.1396. ¹H NMR (500 MHz, CD₃OD) δ 7.85 (d, J=0.8 Hz, H-1, 1H), 7.60-7.53 (m, H-2, H-3, 2H), 7.41 (d, J=1.3 Hz, H-5, 1H), 7.39 (s, H-14, 1H), 7.15 (dd, J=8.2, 1.7 Hz, H-4, 1H), 6.94 (d, J=3.9 Hz, H-13, 1H), 6.21 (s, H-15, 1H), 6.15 (d, J=3.9 Hz, H-12, 1H), 5.21 (d, J=14.1 Hz, H-6, 1H), 4.80 (d, J=14.2 Hz, H-6, 1H), 4.06 (d, J=14.4 Hz, H-7, 1H), 3.84 (d, J=14.4 Hz, H-7, 1H), 3.09 (t, J=7.4 Hz, H-11, 2H), 2.54 (t, J=7.4 Hz, H-10, 2H), 2.50 (s, H-16, 3H), 2.27 (s, H-17, 3H). ¹³C NMR (125 MHz, CD₃OD) δ 175.49, 160.11, 156.59, 144.54, 136.56, 135.16, 134.17, 133.40, 132.28, 130.99, 130.12, 128.09, 126.80, 126.00, 124.35, 120.02, 116.11, 76.97, 54.64, 45.84, 33.87, 29.34, 23.99, 13.47, 9.78. ¹⁹F NMR (470 MHz, CD₃OD) δ-145.27(m, F_{BODIPY}).

### Example 2

### Biological Activity of Probes 1-6

Using the pharmacophore of the 1,2,4-triazole based antifungal agents fluconazole and itraconazole, we designed and synthesized fluorescent probes 3-6 and evaluated their localization in a collection of pathogenic yeast cells. To be effective for use in living cells, a tracker must not affect cell viability at labeling concentration and during the time of the experiment. To reduce fungal cell toxicity, probes 2, 3, 5 and 6 were thus designed with a 1,2,3-triazole instead of a 1,2,4-triazole ring. In triazole-based antifungal azoles, the 1,2,4-triazole ring interacts with the iron atom in the heme of the target enzyme CYP51. As previously shown through density functional theory calculations, the electron density of the nitrogen atom in 1,2,4-triazoles that interacts with the heme iron is 33% higher than that of the corresponding nitrogen atom in 1,2,3-triazoles (Benhamou et al., 2018). We therefore reasoned that isosteric 1,2,3-triazole-based analogs of antifungal azoles bind in the catalytic domain of CYP51 to facilitate specific ER-labeling in fungal cells, but would not significantly affect the catalytic activity of the enzyme. In order to study possible halogen atom effects on the fluorescence, localization, and cell permeability, the difluorophenyl ring segment of probes 1, 2, 4 and 5 was displaced by dichlorophenyl in probes 3 and 6. Furthermore, to extend compatibility of the ER trackers with other fluorescent markers by preventing excitation/emission overlaps, probes 1-3 are based on 7-(diethyl)-aminocoumarin whereas probes 4-6 are BODIPY-based.

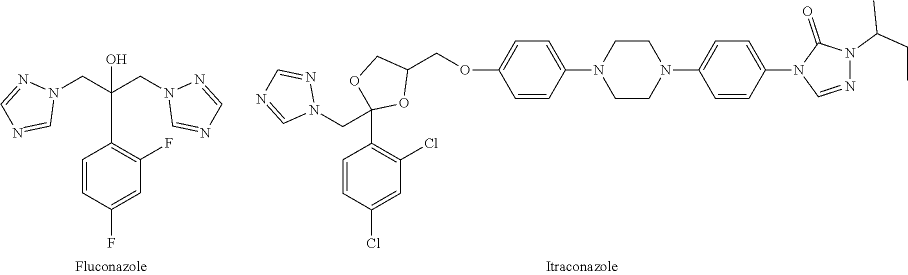

We first measured the absorption and emission spectra of fluorescent probes 1-6. Interestingly, although aminocoumarin-based probes 2 and 3 shared the same absorption spectrum maxima value (430 nm), their emission maxima values differ significantly: 480 nm and 550 nm, respectively. This 70-nm redshift effect is attributed to the effect of the halogen atoms in the phenyl ring segment of these molecules: a difluorophenyl ring in probe 2 and a dichlorophenyl in probe 3. In contrast, no halogen atom effect was observed for the BODIPY-based probes 4, 5 and 6, which shared the same absorption and emission maxima values (ex: 505 nm; em: 515 nm). The differences in the trizaole rings did not affect the absorption or emission spectra.

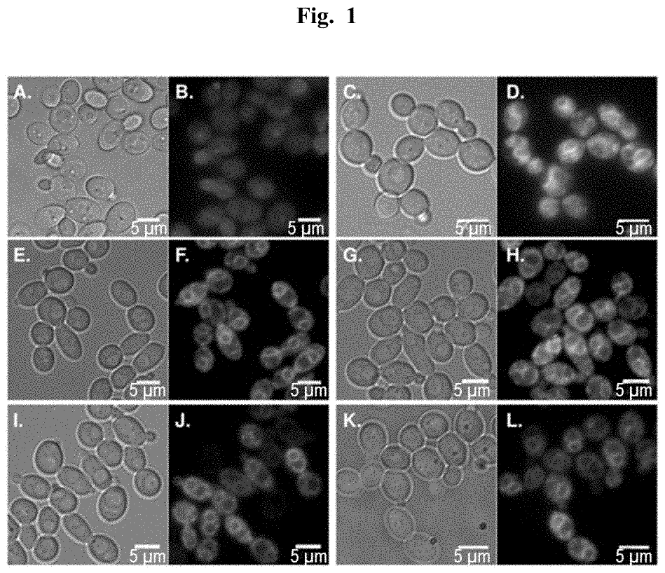

The ER-labeling properties were evaluated by incubating the compounds with representative strains of C. albicans and C. glabrata. These two types of Candida are phylogenetically, genetically, and phenotypically different, and considered the two most common fungal pathogens that cause infections in humans. Since the goal of this study was to develop ER trackers suitable for staining of live cells, we first tested if the synthetic fluorescent azoles displayed antifungal activity against the panel of Candida by determining their MIC values (Table 2). The aminocoumarin-based probe 1, which has a 1,2,4-triazole ring, had potent antifungal activity against all tested Candida strains with MICs ranging from 0.007 to 16 μg/mL. The BODIPY-based probe 4, which has a 1,2,4-triazole ring, had MICs of 0.5-1 μg/mL against the tested C. albicans strains but was inactive against the tested C. glabrata strains (MIC≥64 μg/mL). Probes 2, 3, 5 and 6, which contain a 1,2,3-triazole ring, had no antifungal activity at the highest concentration tested (MIC≥64 μg/mL). These azoles were therefore further evaluated as fungal ER trackers in live cell imaging experiments with four Candida strains (FIG. 1 and FIGS. 5-8). The commercial ER-trackers BODIPY TR Glibenclamide (ex: 587 nm; em: 615 nm), Blue-White DPX (ex: 374 nm; em: 430-640 nm) and DiOC₆ (Koning et al., 1993) (ex: 480 nm; em: 500 nm) were used as controls.

TABLE 2
Antifungal activity of parent azoles antifungal drugs FLC and ITR, and
fluorescent azoles 1-6

| | | | MIC [μg/mL] | | | | | |
|---|---|---|---|---|---|---|---|---|
| Yeast strain | FLC | ITR | 1 | 2 | 3 | 4 | 5 | 6 |
| C. albicans ATCC 24433 | 0.5 | 0.015 | 0.03 | >64 | >64 | 1 | >64 | >64 |
| C. albicans SN152 | 0.5 | 0.007 | 0.007 | >64 | >64 | 0.5 | >64 | >64 |
| C. glabrata ATCC 66032 | 64 | 0.5 | 8 | >64 | >64 | >64 | >64 | >64 |
| C. glabrata ATCC 2001 | 64 | 4 | 16 | >64 | >64 | >64 | >64 | >64 |

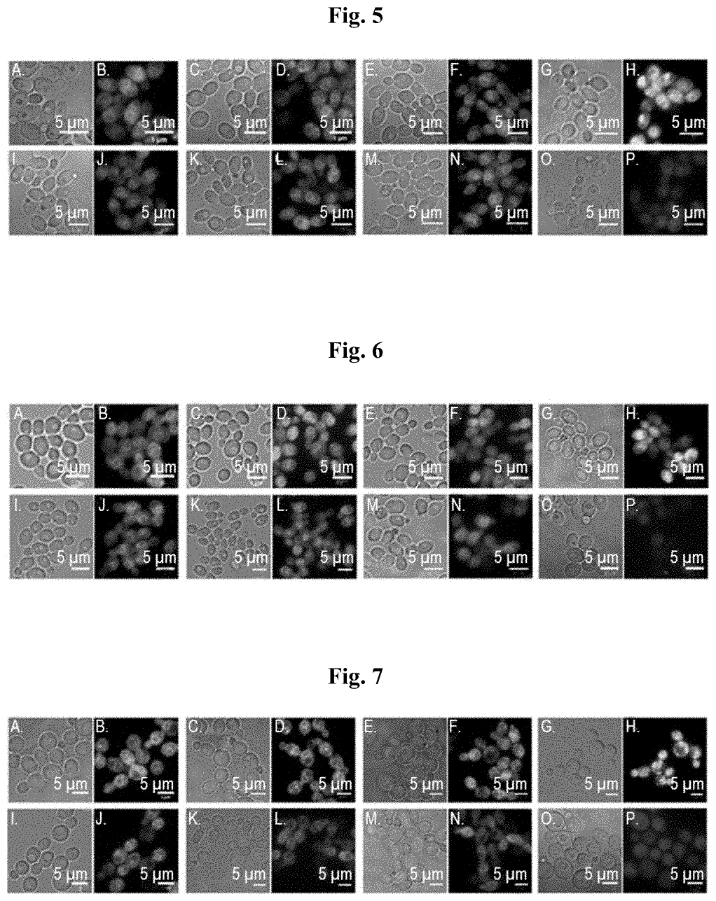

In yeast cells, the ER forms a distinct circular pattern around the nuclear envelope, which extends in branches that occupy the cytoplasm and termed the peripheral ER. As in cells of higher eukaryotes, the peripheral ER in yeast cells is mainly organized into a membrane network adjacent to the inner leaflet of the plasma membrane; the ER region near the plasma membrane is referred to as the cortical ER. To optimize the probes, we investigated the effects of the concentration, time of incubation, and staining medium (for optimal staining conditions, see Experimental section). In C. albicans cells incubated with the commercial ER tracker Blue-White DPX, we observed non-specific staining of the cytoplasm (FIG. 1, panels A-B, and FIG. 7). Blue-White DPX did, however, label the ER in yeast cells of the C. glabrata strains (FIGS. 5-6). Although a staining pattern expected for the ER was observed when DiOC₆ was incubated with C. albicans strain SN152, non-specific staining of other cellular compartments such as lipid droplets was also observed (FIG. 1, panels C-D). Non-specific labeling of organelles by DiOC₆ was more significant in C. albicans than in C. glabrata yeast cells (FIG. 1, panel D, and FIGS. 5-7). Under the conditions screened, the commercial ER tracker BODIPY TR Glibenclamide did not stain the interior of any of the tested Candida strains (FIG. 10).

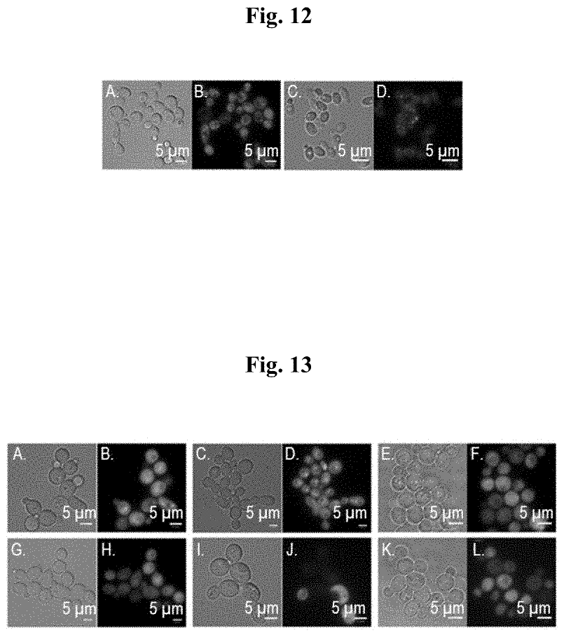

When cells of the tested panel of Candida strains were stained with probes 2, 3, 5 and 6, the distinct nuclear envelope and cortical structure of the ER were clearly observed (FIG. 1, panels E-L, and FIGS. 5-7). Finally, the ethyl ester of 7-(diethyl)-aminocoumarin, which is the fluorophore segment of probes 1-3, did not stain the yeast cell interior of any of the tested Candida strains (FIG. 11); in yeast cells labeled with only the BODIPY-methyl ester, the fluorophore segment of probes 4-6, the entire cytoplasm was labeled in a non-specific pattern (FIG. 12). This demonstrates that ER-labeling specificity of the fluorescent azoles does not result from their fluorescent dye segment.

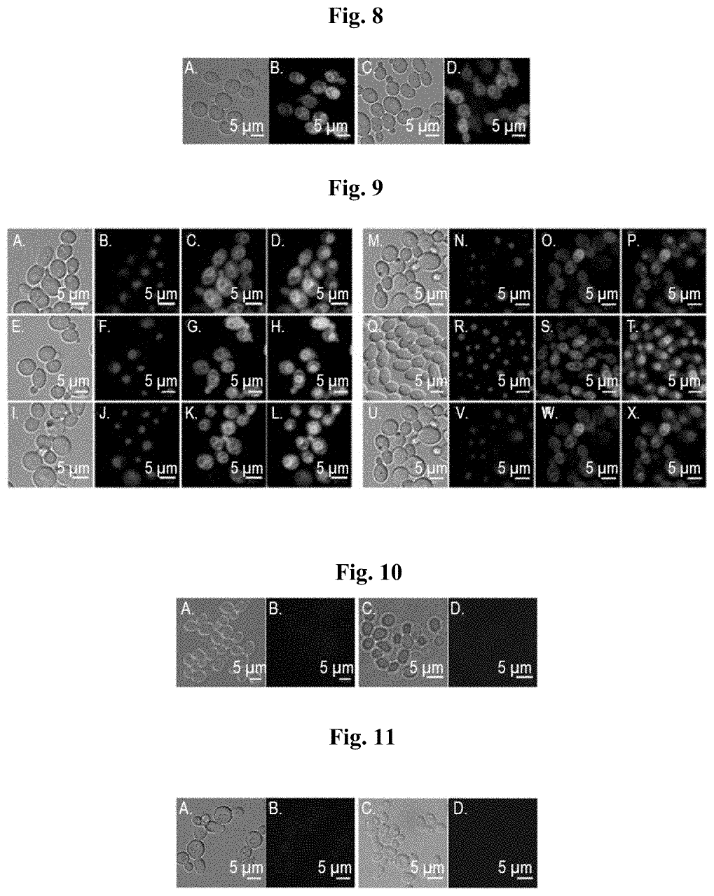

Since an ER-pattern was observed in C. glabrata yeast cells that were stained with Blue-White DPX but not in C. albicans, we treated C. glabrata with probes 5 and ER-tracker Blue-White DPX; probe 5 was chosen since the excitation/emission wavelengths of this compound and of Blue-White DPX do not coincide. In C. glabrata cells stained with Blue-White DPX, a circular pattern around the nuclear envelope with peripheral cortical extensions reminiscent of the ER structure was clearly visible (FIG. 2, panels A-B). High co-localization was observed between the subcellular staining patterns of probe 5 and that of the Blue-White DPX (FIG. 2, panels A-D) with a Pearson correlation coefficient of 0.86±0.04 further supporting the ER-selectivity of the fluorescent azoles. Next, we determined the localization of probe 5 in yeast cells of a C. albicans strain that expresses mCherry-labeled Eno1, a protein that localizes largely to the nucleus. The fluorescent pattern of probe 5 surrounded the Eno1-mCherry-labeled nuclei as expected of an ER localization which circles the nuclear envelope (FIG. 2, panels E-H, and FIG. 9).

To further study the ER specificity of the fluorescent azoles, we compared the labeling patterns in a C. albicans mutant strain lacking copies of both the ERG11 and ERG3 genes to that of strain SN152 from which the mutant strain was derived (Table 3). ERG11 encodes CYP51, the target of antifungal azoles that is essential for fungal cell growth. The C-5 sterol desaturase encoded by ERG3 is necessary for CYP51 function. While the characteristic nuclear envelope and cortical ER pattern appeared in cells of the parental C. albicans SN152 strain that were labeled with azole 5, no ER-labeling pattern appeared in cells lacking CYP51 and the entire cytoplasm of these cells was stained (FIG. 2, panels K-L, and FIG. 13). This supports that the reason for the ER specificity of the azole fluorescent probes results from the pharmacophore of the azole class of antifungal drugs that binds to CYP51, which localizes primarily to the ER in yeast cells.

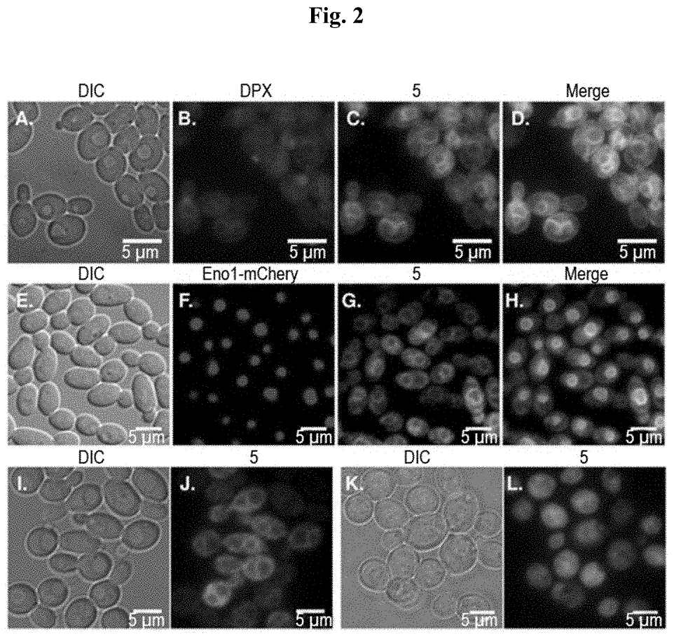

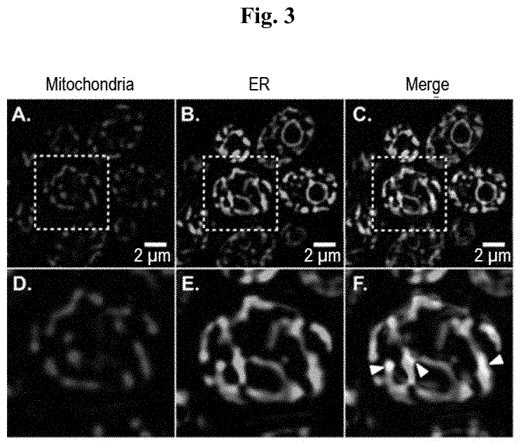

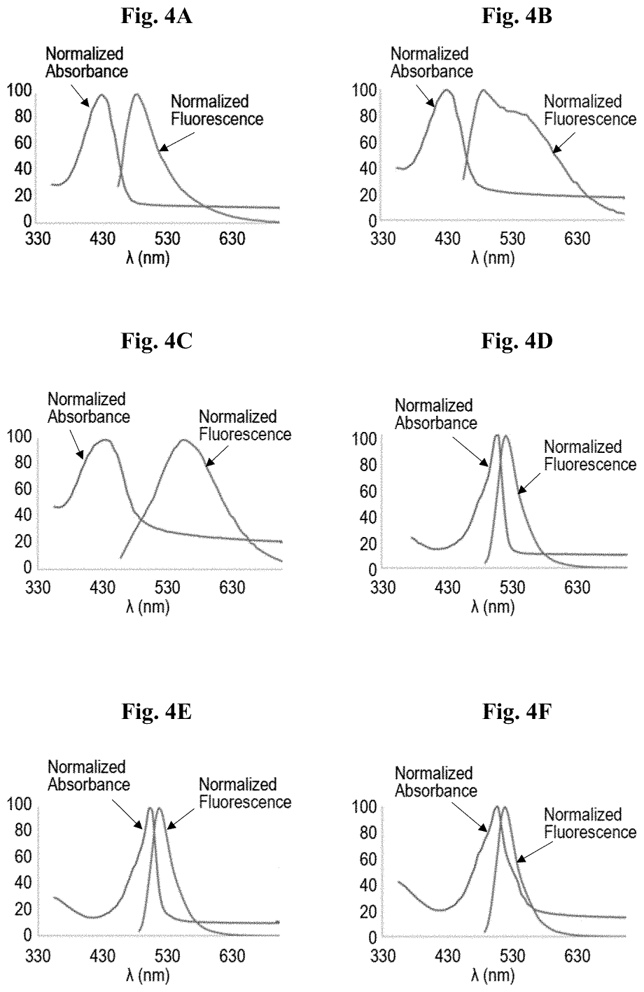

Using the fluorescent ER trackers disclosed herein, it should be possible to detect interconnections between the ER and other organelles by simply staining the fungal cells with a combination of organelle trackers. This strategy offers a robust protocol for studying organelle interconnections and saves the need for cloning cells with fluorescent reporter proteins. It is now well accepted that inter-organelle communication occurs via membrane contact sites (MCSs) (Wu et al., 2018). At these sites, organelles are tethered together in close proximity to facilitate various inter-organelle functions. MCSs between the ER and mitochondria are involved in calcium signaling, lipid biosynthesis, and mitochondrial division (Martinvalet, 2018). Contacts between the ER and mitochondria were previously visualized by confocal fluorescence microscopy in live cells. In those studies, fluorescent labeling of the ER and mitochondria was accomplished using organelle-specific fluorescent reporter proteins and/or immunoblotting (Valm et al., 2017). The present study demonstrates that both visualization of the intracellular organization of the ER and mitochondria and identification of MCSs between these two organelles in Candida cells were possible by staining of the cells with a combination of probe 3 and a mitochondrial dye composed of a Cy5-based antifungal azole previously developed in our group (FIG. 3, panels A-B) (Benhamou et al., 2017). Merged images of the fluorescently labeled mitochondria and ER revealed sites that can be attributed to MCSs where the characteristic uniform network of mitochondrial tubules co-localizes with the ER (FIG. 3, panel C). Similar co-localization sites were previously observed between green fluorescent protein (GFP)-labeled ER and red fluorescent protein (RFP)-labeled mitochondria in yeast cells of Saccharomyces cerevisiae using confocal microscopy, and were attributed to tethering sites between the two organelles (Vanessa Saura et al., 2017).

TABLE 3
Candida strains used in this study

| No. | Species | Strain name | | Genome | Parent Strain | Comment |
|---|---|---|---|---|---|---|
| A¹ | C. albicans | | ATCC 24433 | | | |
| B² | C. albicans | SN152 | Parental | eu2Δ/leu2Δhis1Δ/his1Δ | SN148 | |
| | | | | arg4Δ/arg4Δ | | |
| | | | | URA3/ura3Δ:imm434 | | |
| | | | | IRO1/iro1Δ:imm434 | | |
| C³ | C. glabrata | 660 | ATCC 2001 | — | | WT |
| D¹ | C. glabrata | | 66032 | — | | WT |
| E² | C. albicans | BV11 | ΔΔerg3/ | leu2Δ/leu2Δhis1Δ/his1Δ | SN152 | Fluconazole |
| | | | ΔΔerg11 | arg4Δ/arg4Δ | | resistant |
| | | | | URA3/ura3Δ:imm434 | | strain |
| | | | | IRO1/iro1Δ:imm434 | | |
| | | | | erg3Δ::C.d.HIS1/erg3Δ: | | |
| | | | | C.m.LEU2 | | |
| | | | | erg11Δ:C.d.ARG4/ | | |
| | | | | erg11Δ/C.d.ARG4 | | |
| F³ | C. albicans | YJB11257 | Eno1-mCherry | ura3Δ:λimm434/ura3Δ:λim | BWP17 | |
| | | | | m434 his1:hisG/his1::hisG | | |
| | | | | arg4:hisG/arg4:hisG | | |
| | | | | Eno1-mCherry-URA3 | | |

¹obtained from ATCC
²obtained from Susan Lindquist; and
³obtained from Judith Berman.

### APPENDIX

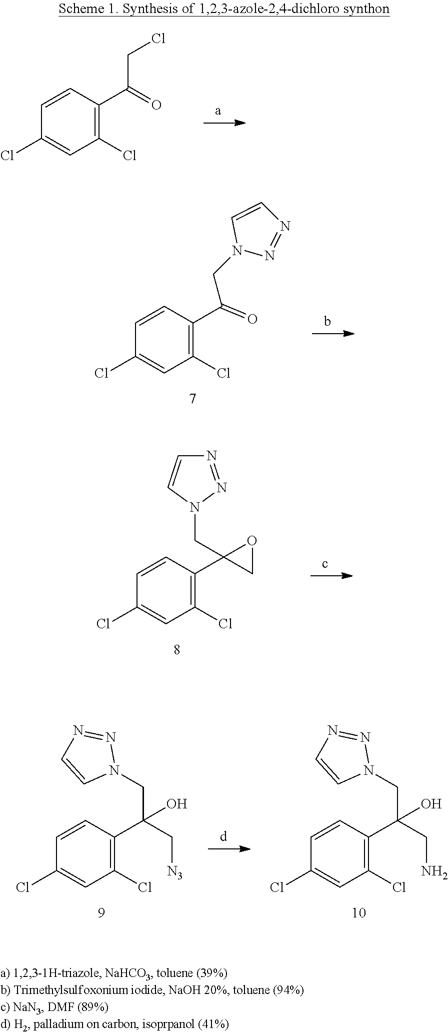 [SMILES: O=C(CCl)c1ccc(Cl)cc1Cl ; O=C(Cn1ccnn1)c1ccc(Cl)cc1Cl ; Clc1ccc(C2(Cn3ccnn3)CO2)c(Cl)c1 ; [N-]=[N+]=NCC(O)(Cn1ccnn1)c1ccc(Cl)cc1Cl ; NCC(O)(Cn1ccnn1)c1ccc(Cl)cc1Cl]

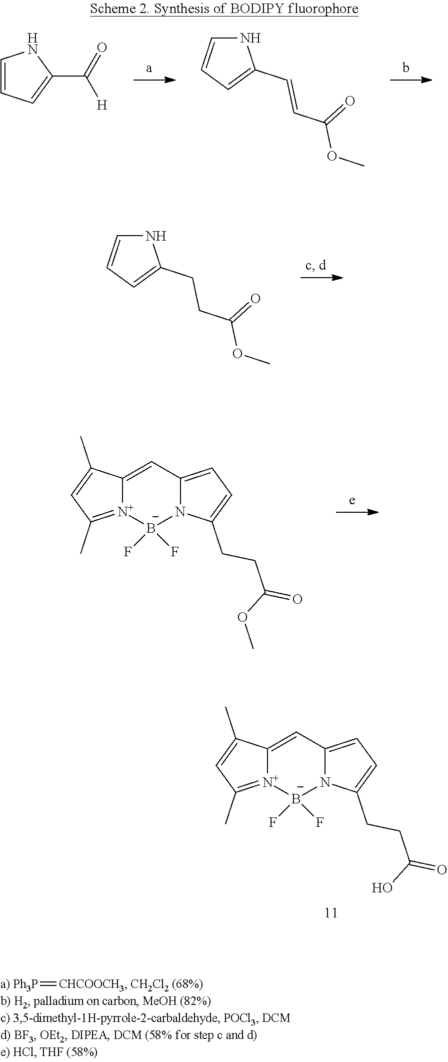

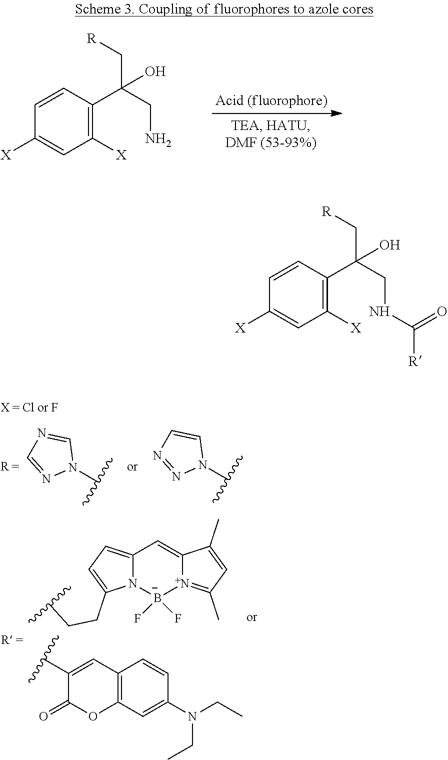

### REFERENCES

Benhamou, R. I.; Bibi, M.; Steinbuch, K. B.; Engel, H.; Levin, M.; Roichman, Y.; Berman, J.; Fridman, M. Real-time imaging of the azole-class of antifungal drugs in live candida cells. ACS Chem. Biol. 2017, 12, 1769-1777

Benhamou, R. I.; Bibi, M.; Berman, J.; Fridman, M. Localizing antifungal drugs to the correct organelle can markedly enhance their efficacy. Angew. Chemie Int. Ed. 2018, 57, 6230-6235

Fernández-Suárez, M.; Ting, A. Y. Fluorescent probes for super-resolution imaging in living cells. Nat. Rev. Mol. Cell Biol. 2008, 9, 929-943

Kim, D.; Liu, Y.; Benhamou, R. I.; Sanchez, H.; Simon-Soro, A.; Li, Y.; Hwang, G.; Fridman, M.; Andes, D. R.; Koo, H. Bacterial-derived exopolysaccharides enhance antifungal drug tolerance in a cross-kingdom oral biofilm. ISME J. 2018, 12, 1427-1442

Koning, A. J.; Lum, P. Y.; Williams, J. M.; Wright, R. DiOC₆ staining revels organelle structure and dynamics in living yeast cells. Cell Motil.Cytoskeleton. 1993, 25, 111-128

Kornmann, B.; Currie, E.; Collins, S. R.; Schuldiner, M.; Nunnari, J.; Weissman, J. S.; Walter, P. An ER-mitochondria tethering biology screen. Science. 2009, 325, 477-481

Loudet, A.; Burgess, K. BODIPY dyes and their derivatives: syntheses and spectroscopic properties, Chem. Rev. 2007, 107, 4891-4932

Martinvalet, D. The role of the mitochondria and the endoplasmic reticulum contact sites in the development of the immune responses. Cell Death Dis. 2018, 9, 336

Valm, A. M.; Cohen, S.; Legant, W. R.; Melunis, J.; Hershberg, U.; Wait, E.; Cohen, A. R.; Davidson, M. W.; Betzig, E.; Lippincott-Schwartz, J. Applying systems-level spectral imaging and analysis to reveal the organelle interactome. Nature 2017, 546, 162-167

Vanessa Saura, A.; Isabel Burguete, M.; Galindo, F.; Luis, S. V.; Xu, Y.; Li, H.; Wang, C.; Lu, A.; Sun, S.; Burgess, K. Novel fluorescent anthracene-bodipy dyads displaying sensitivity to pH and turn-on behaviour towards Cu(II) ions. Org. Biomol. Chem. 2017, 15, 3013-3024

Wu, H.; Carvalho, P.; Voeltz, G. K. Here, there, and everywhere: the importance of ER membrane contact sites. Science. 2018, 361, eaan5835

Xu, W.; Zeng, Z.; Jiang, J. H.; Chang, Y. T.; Yuan, L. Discerning the chemistry in individual organelles with small-molecule fluorescent probes. Angew. Chemie Int. Ed. 2016, 55, 13658-13699

Zhang, J.; Campbell, R. E.; Ting, A. Y.; Tsien, R. Y. Creating new fluorescent probes for cell biology. Nat. Rev. Mol. Cell Biol. 2002, 3, 906-918

What is claimed is:

1. A compound of the formula I: 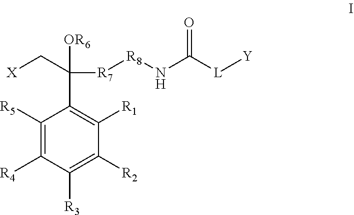
wherein:
X is selected from the group consisting of 1,2,3-triazole optionally substituted with one or two F atoms, and 1,2,4-triazole substituted with one or two F atoms, and attached via a nitrogen atom thereof to the adjacent —CH₂—;
L is absent, or a linker selected from the group consisting of (C₁-C₆)alkylene, (C₂-C₆)alkenylene, and (C₂-C₆)alkynylene, and optionally interrupted with an aromatic or aliphatic ring;
Y is a fluorescent dye selected from the group consisting of boron-dipyrromethene (BODIPY) and coumarin, attached via a carbon atom thereof and optionally substituted with at least one substituent each independently selected from the group consisting of (C₁-C₆)alkyl, —O—(C₁-C₆)alkyl, Cl, Br, I, —CN, and —N(R′)₂ wherein R′ each independently is H, (C₁-C₆)alkyl, (C₂-C₆)alkenyl, or (C₂-C₆)alkynyl, or the two R′ s together with the N atom to which they are attached form 5- or 6-membered heterocyclic ring optionally containing further 1 or 2 heteroatoms selected from N, O and S, provided that when Y is substituted with two (C₁-C₆)alkyl groups at two adjacent carbon atoms thereof, said (C₁-C₆)alkyl groups together with the carbon atoms to which they are attached may form a 5- or 6-membered carbocyclic ring;
R₁ , R_{2,} R_{3,} R_{4,} and R₅ each independently is selected from the group consisting of H, halogen, —CN, and a heteroaryl containing N atom and optionally at least one further heteroatom selected from N, O and S;
R₆ is H or —CH₂—;
R₇ is —CH₂— or —O—CH₂—CH₂—O—; and
R₈ is absent, arylene-diyl, or (C₁-C₆)alkylene,
provided that when R₆ is H, R₇ is —CH₂—; and when R₆ is —CH₂—, R₇ is —O—CH₂—CH₂—O—, and R₆ together with one of the carbon atoms of R₇ form a 5- or 6-membered heterocyclic ring.

2. The compound of claim 1, wherein X is 1,2,3-triazole, or 1,2,4-triazole substituted with one or two F atoms.

3. The compound of claim 1, wherein R_{1,} R_{2,} R_{3,} R_{4,} R₅ each independently is selected from the group consisting of H, F, and Cl.

4. The compound of claim 3, wherein R_{3,} and one of R₁ and R_{5,} each is F or Cl;
and R_{2,} R_{4,} and the other one of R₁ and R₅ each is H.

5. The compound of claim 1, wherein R₆ is H; and R₇ is —CH₂—.

6. The compound of claim 5, wherein R₈ is absent.

7. The compound of claim 1, wherein R₆ is —CH₂—; R₇ is —O—CH₂—CH₂—O—; and R₆ together with one of the carbon atoms of R₇ form a 5-membered heterocyclic ring.

8. The compound of claim 7, wherein R₈ is (C₁-C₆)alkylene or arylene-diyl such as phenylene.

9. The compound of claim 1, wherein L is absent, or (C₁-C₆)alkylene optionally interrupted with an aromatic or aliphatic ring.

10. The compound of claim 1, wherein Y is coumarin substituted with —N(R′)_{2,} wherein R′ is (C₁-C₃)alkyl; or BODIPY substituted with two identical or different (C₁-C₃)alkyl groups.

11. The compound of claim 10, wherein Y is 7-diethylaminocoumarin such as 7-(diethylamino)-coumarin-3-yl; or 4,4-difluoro-1,4-dimethyl-4-bora-3a,4a-diaza-s-indacen-5-yl.

12. The compound of claim 1, wherein:
X is 1,2,3-triazole, or 1,2,4-triazole substituted with one or two F atoms;
R_{1,} R_{2,} R_{3,} R_{4,} R₅ each independently is selected from the group consisting of H, F, and Cl;
L is absent, or (C₁-C₆)alkylene optionally interrupted with an aromatic or aliphatic ring; and
Y is coumarin substituted with —N(R′)_{2,} wherein R′ is (C₁-C₃)alkyl; or BODIPY substituted with two identical or different (C₁-C₃)alkyl groups.

13. The compound of claim 12, wherein:
R_{3,} and one of R₁ and R_{5,} each is F or Cl; and R_{2,} R_{4,} and the other one of R₁ and R₅ each is H;
L is absent, or (C₁-C₄)alkylene; and
Y is 7-diethylaminocoumarin such as 7-(diethylamino)-coumarin-3-yl; or 4,4-difluoro-1,4-dimethyl-4-bora-3a,4a-diaza-s-indacen-5-yl.

14. The compound of claim 13, wherein R₆ is H; and R₇ is —CH₂—.

15. The compound of claim 14, wherein R₈ is absent.

16. The compound of claim 15, wherein X is 1,2,3-triazole; R₁ and R₃ each is F or Cl; R_{2,} R_{4,} and R₅ each is H; R₆ is H; R₇ is —CH₂—; R₈ is absent; and:
(i) L is absent; and Y is 7-(diethylamino)-coumarin-3-yl: 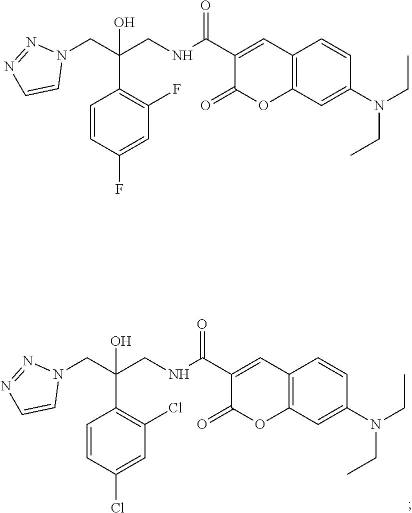 [SMILES: CCN(CC)c1ccc2cc(C(=O)NCC(O)(Cn3ccnn3)c3ccc(F)cc3F)c(=O)oc2c1 ; CCN(CC)c1ccc2cc(C(=O)NCC(O)(Cn3ccnn3)c3ccc(Cl)cc3Cl)c(=O)oc2c1]
or (ii) L is —(CH₂)₂—; and Y is 7-(diethylamino)-coumarin-3-yl.

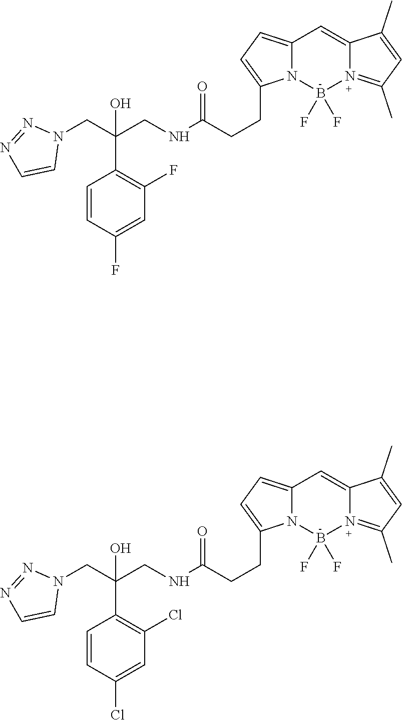

17. The compound of claim 13, wherein R₆ is —CH₂—; R₇ is —O—CH₂—CH₂—O—; and R₆ together with one of the carbon atoms of R₇ form a 5-membered heterocyclic ring.

18. The compound of claim 17, wherein R₈ is absent or arylene-diyl such as phenylene.

19. A composition comprising a compound according to claim 1.

20. The composition of claim 19, wherein X is 1,2,3-triazole; R₁ and R₃ each is F or Cl; R_{2,} R_{4,} and R₅ each is H; R₆ is H; R₇ is —CH₂—; R₈ is absent; and:
(i) L is absent; and Y is 7-(diethylamino)-coumarin-3-yl; or (ii) L is —(CH₂)₂-; and Y is 7-(diethylamino)-coumarin-3-yl.

21. A method for tracking the endoplasmic reticulum in a fungal cell, said method comprising incubating said fungal cell with a compound according to claim 1 or a composition comprising said compound; irradiating said fungal cell with a light at a wavelength within the excitation spectrum of said compound; and imaging the light emitted from said compound.
